(19) United States
(12) Patent Application Publication
(10) Pub. No.: US 2024/0181232 A1
(43) Pub. Date: Jun. 6, 2024

(54) BODY CHAMBER THERAPEUTIC SUBSTANCE DELIVERY

(71) Applicant: Cochlear Limited, Macquarie University, NSW (AU)

(72) Inventors: Daniel Smyth, Dublin (IE); Wolfram Frederik Dueck, Berlin (DE)

(21) Appl. No.: 18/553,787

(22) PCT Filed: Mar. 18, 2022
(86) PCT No.: PCT/IB2022/052497

§ 371 Date: Oct. 3, 2023

Related U.S. Application Data
(60) Provisional application No. 63/176,933, filed on Apr. 20, 2021.

(51) Int. Cl. A61M 31/00; A61F 11/00

(52) U.S. Cl. CPC A61M 31/002; A61F 11/00; A61M 2210/0668

(57) ABSTRACT

Presented herein are therapeutic substance delivery devices configured to be inserted into a body chamber of a recipient. The therapeutic substance delivery devices are configured to assume a position in close proximity to a first wall of the body chamber, without affecting body structures located at a second (opposing) side of body chamber.

### BACKGROUND

### Field of the Invention

The present invention relates generally to the implantable delivery of therapeutic substances to a body chamber in a recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: inserting a therapeutic substance delivery device into a body chamber of a recipient; adhering the therapeutic substance delivery device to a surface of the body chamber; and delivering, with the therapeutic substance delivery device, one or more therapeutic substances to the body chamber.

In another aspect, an apparatus is provided. The apparatus comprises: a fully bioresorbable substrate configured to be inserted into a body chamber of a recipient to assume a position in close proximity to a first side of the body chamber without affecting structures of the body chamber on a second side of the body chamber; and one or more therapeutic substances disposed in or on the substrate.

In another aspect, a method is provided. The method comprises: forming an opening in a tissue barrier associated with a body chamber in a body of a recipient; inserting an elongate applicator into the body chamber via the opening, wherein a therapeutic substance delivery device is attached to the applicator; positioning the therapeutic substance delivery device abutting a first wall of the body chamber; releasing the therapeutic substance delivery from the applicator; and withdrawing the elongate applicator from the elongate body chamber via the opening.

In another aspect, an apparatus is provided. The apparatus comprises: an elongate substrate formed from an adhesive and bioresorbable material and configured to be inserted into a body chamber of a recipient to assume a position in close proximity to a first side of the body chamber without affecting structures of the body chamber on a second side of the body chamber, wherein the adhesive material is configured to adhere to a wet surface at the first side of the body chamber, wherein the substrate has a porous structure and the substrate has a thickness and a width, wherein the width is substantially greater than the thickness, wherein the width of the substrate is at least twice the thickness of the substrate, wherein the substrate has a pre-curved shape and is configured to be held straight prior to insertion into the body chamber, wherein during insertion into the body chamber the substrate is configured to return to the pre-curved shape, and one or more therapeutic substances disposed in or on the substrate.

### BRIEF DESCRIPTION OF THE DRAWINGS

Embodiments of the present invention are described herein in conjunction with the accompanying drawings, in which:

FIGS. 1A, 1B, and 1C are schematic diagrams illustrating the cochlea of a recipient in which therapeutic substance delivery devices in accordance with certain embodiments presented herein can be implanted;

FIGS. 2A and 2B are schematic diagrams illustrating a therapeutic substance delivery device, in accordance with certain embodiments presented herein;

FIG. 3 is a schematic diagram illustrating another therapeutic substance delivery device, in accordance with certain embodiments presented herein;

FIGS. 4A, 4B, and 4C are schematic diagrams illustrating a therapeutic substance delivery device, in accordance with certain embodiments presented herein;

FIGS. 5A, 5B, 5C, and 5D are schematic diagrams illustrating another therapeutic substance delivery device, in accordance with certain embodiments presented herein;

FIG. 6 is a schematic diagram illustrating another therapeutic substance delivery device, in accordance with certain embodiments presented herein;

FIG. 7A is a side-view of a portion of another therapeutic substance delivery device, in accordance with certain embodiments presented herein;

FIG. 7B is a side-view of a portion of another therapeutic substance delivery device, in accordance with certain embodiments presented herein;

FIG. 7C is a top-view of a portion of another therapeutic substance delivery device, in accordance with certain embodiments presented herein;

FIG. 8 is a flowchart illustrating an example method, in accordance with certain embodiments presented herein;

FIG. 9 is a flowchart illustrating another example method, in accordance with certain embodiments presented herein;

FIG. 10 is a flowchart illustrating another example method, in accordance with certain embodiments presented herein; and

FIG. 11 is a schematic diagram illustrated a therapeutic substance delivery device, in accordance with certain embodiments presented herein, implanted proximate to a retina of a recipient.

### DETAILED DESCRIPTION

A growing area of research and development relates to the use of pharmaceutical compounds, biological substances, bioactive substances, etc., including pharmaceutical agents/active pharmaceutical ingredients (APIs), genes, messenger RNA (mRNA) or other signalling compounds that promote recovery and resolution, chemicals, ions, drugs, etc. to treat a variety of disorders within the body of individual patient/recipient. These various substances, which are collectively and generally referred to herein as “therapeutic substances,” are delivered to induce some therapeutic results/treatment within the body of the recipient. For example, therapeutic substances may be delivered to treat ear disorders (e.g., tinnitus, hearing loss, tinnitus, Ménière's disease, etc.), to treat infections post-surgery, to fight cancer cells, to treat neurodegenerative diseases, to treat infectious diseases, etc.

The body of an animal, including the body of a human recipient (“recipient”), includes a number of different body chambers. In certain examples, these body chambers are fluidically-sealed (e.g., cavities or enclosed areas in which bodily fluids are sealed). For example, sensitive tissues in the body of a recipient, such as the brain, the ear, the eye, etc. are protected from the normal circulation by fluidic tissue barriers. In particular, the brain is surrounded by the blood-brain barrier (BBB), the inner ear (including the cochlea and the vestibular system) are surrounded by the blood-labyrinth barrier (BLB), the eye retina is surrounded by the blood-ocular barrier (BOB), which includes the blood-aqueous barrier (BAB) and the blood-retinal barrier (BRB), and so on. Other tissue barriers, such as the round window, and/or the oval window, are also present in the body of a recipient and are two tissue barriers associated with a fluidically-sealed cochlea of a recipient.

As noted, it can be advantageous to deliver therapeutic substances to the body of a recipient, including various body chambers. However, using conventional techniques, it is difficult to deliver therapeutic substances to certain body chambers (e.g., fluidically-sealed chambers) within the body of a recipient without compromising the near-term or long-term structural and functional integrity of certain structures within the chamber. As such, presented herein are devices configured to deliver therapeutic substances, in an atraumatic manner, to a chamber within the body of a recipient (e.g., a “body chamber”). As described elsewhere herein, therapeutic substance delivery devices in accordance with certain embodiments presented herein can be used to deliver therapeutic substances to any of a number of body chambers located, for example, behind a number of different tissue barriers, including, fluidically-sealed body chambers located behind the blood-brain barrier (BBB), behind the blood-labyrinth barrier (BLB), behind the blood-ocular barrier (BOB), which includes the blood-aqueous barrier (BAB) and the blood-retinal barrier (BRB), and so on. For example, therapeutic substance delivery devices in accordance with certain embodiments presented herein can be used to deliver therapeutic substances the scala tympani, the scala media, the scala vestibuli, the semi-circular canals, any other volume of the labyrinthine, the retina, etc.

More specifically, therapeutic substance delivery devices presented herein comprise a delivery substrate configured to be inserted into a body chamber of a recipient. The therapeutic substance delivery devices are configured to assume a position in close proximity to a first wall of the body chamber, without affecting body structures located at a second (opposing) side of body chamber and/or structures located at walls connecting the first and second walls. The delivery substrate is configured to deliver one or more therapeutic substances to the body chamber. For example, the delivery substrate can include (e.g., have disposed therein or thereon) one or more therapeutic substances such that, following insertion of the substrate into the elongate body chamber, the one or more therapeutic substances elute into the body chamber.

Merely for ease of description, the therapeutic substance delivery devices presented herein will primarily be described with reference to the delivery of therapeutic substances to a specific body chamber in a recipient, namely the cochlea of a recipient. As described below, in the cochlea, the therapeutic substance delivery devices are generally configured to assume a perimodiolar position (e.g., close to the modiolar wall) and are arranged (e.g., sized/shaped) to be spaced from the lateral wall (e.g., spiral ligament) and, in some anatomy, the stria vascularis, the basilar membrane, the organ of corti, and/or the cochlear aqueduct. However, it is to be appreciated that the therapeutic substance delivery devices presented herein can be used to deliver therapeutic substances to other body chambers and are spaced from other structures therein.

It is also to be appreciated that the therapeutic substance delivery devices presented herein can be used alone or in combination with a number of different types of implantable medical devices. For example, the techniques presented herein may be implemented with auditory prostheses, such as middle ear auditory prostheses, bone conduction devices, direct acoustic stimulators, electro-acoustic prostheses, auditory brain stimulators, cochlear implants, combinations or variations thereof, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

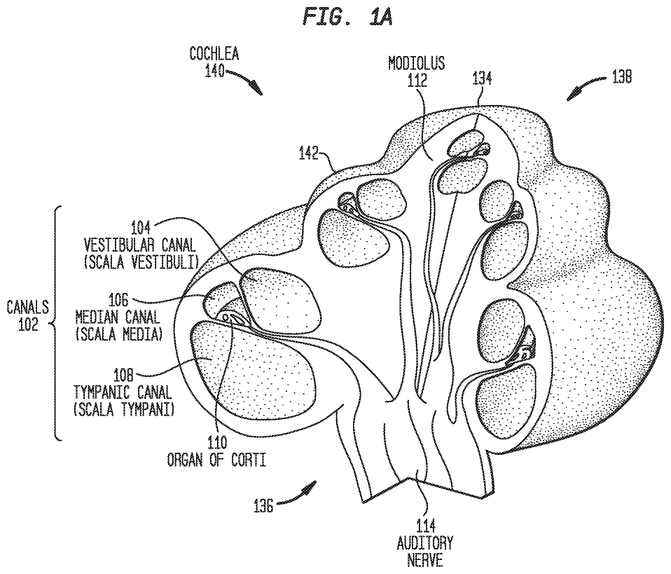

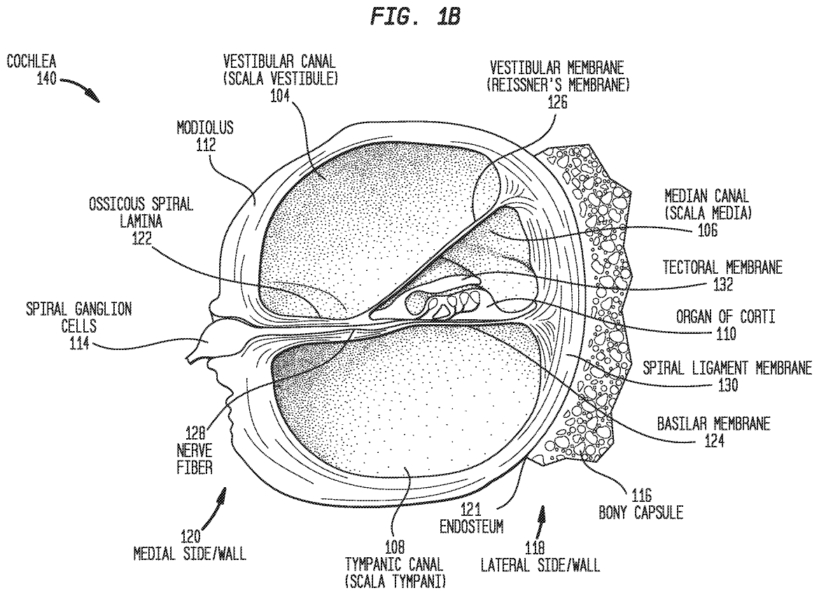

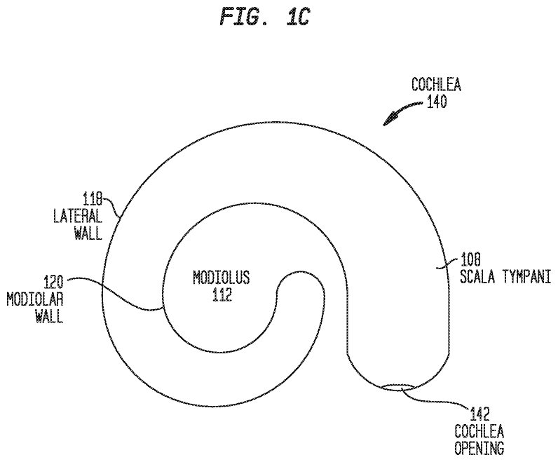

As noted, the therapeutic substance delivery devices are primarily described herein with reference to the delivery of therapeutic substances to the cochlea of a recipient. Before describing details of the therapeutic substance delivery devices, relevant aspects of an example cochlea, in which a therapeutic substance delivery device may be implanted, are first described below with reference to FIGS. 1A, 1B, and 1C. More specifically, FIG. 1A is a perspective view of the cochlea 140 partially cut-away to display the canals and nerve fibers of the cochlea, FIG. 1B is a cross-sectional view of one turn of the canals of the cochlea 140, and FIG. 1C is a simplified cross-sectional view of a portion of cochlea 140.

Referring first to FIG. 1A, cochlea 140 is a conical spiral structure comprising three parallel fluid-filled canals or ducts, collectively and generally referred to herein as canals 102. Canals 102 comprise the tympanic canal 108, also referred to as the scala tympani 108, the vestibular canal 104, also referred to as the scala vestibuli 104, and the median canal 106, also referred to as the scala media 106. The canals 102 of cochlea 140 spiral about modiolus 112 several times and terminate at cochlea apex 134. The scala tympani 108, in particular, is referred to as including a medial or modiolar side/wall 120.

Portions of cochlea 140 are encased in a bony labyrinth/capsule 116 and the endosteum 121 (e.g., a thin vascular membrane of connective tissue that lines the inner surface of the bony tissue that forms the medullary cavity of the bony labyrinth). Spiral ganglion cells 114 reside adjacent the opposing modiolar wall 120 (the left side as illustrated in FIG. 1B) of cochlea 140. A spiral ligament membrane 130 is located between lateral wall 118 of spiral tympani 108 and bony capsule 116, and between lateral wall 118 of scala media 106 and bony capsule 116. Spiral ligament 130 also typically extends around at least a portion of lateral side 118 of scala vestibuli 104.

The fluid in the scala tympani 108 and the scala vestibuli 104, referred to as perilymph, has different properties than that of the fluid which fills scala media 106 and which surrounds organ of Corti 110, referred to as endolymph. The scala tympani 108 and the scala vestibuli 104 collectively form the perilymphatic fluid space 109 of the cochlea 140. Sound entering a recipient's auricle (not shown) causes pressure changes in cochlea 140 to travel through the fluid-filled scala tympani 108 and the scala vestibuli 104. As noted, the organ of Corti 110 is situated on basilar membrane 124 in the scala media 106 and contains rows of 16,000-20,000 hair cells (not shown) which protrude from its surface. Above them is the tectoral membrane 132 which moves in response to pressure variations in the fluid-filled tympanic and vestibular canals 108, 104. Small relative movements of the layers of membrane 132 are sufficient to cause the hair cells in the endolymph to move thereby causing the creation of a voltage pulse or action potential which travels along the associated nerve fiber 128. Nerve fibers 128, embedded within the spiral lamina 122, connect the hair cells with the spiral ganglion cells 114 which form auditory nerve 114. Auditory nerve 114 relays the impulses to the auditory areas of the brain (not shown) for processing.

The place along basilar membrane 124 where maximum excitation of the hair cells occurs determines the perception of pitch and loudness according to the place theory. Due to this anatomical arrangement, cochlea 140 has characteristically been referred to as being “tonotopically mapped.” That is, regions of cochlea 140 toward basal region 136 are responsive to high frequency signals, while regions of cochlea 140 toward apical region 138 are responsive to low frequency signals. In general, the basal region 136 is the portion of the cochlea 140 located closest to the stapes (not shown in FIGS. 1A-1C) and extends to approximately the first turn of the cochlea (i.e., the region of the cochlea 140 between the cochlea openings, including the round and oval windows, the first cochlea turn). The apical region 138 is portion of the cochlea 140 in proximity to the cochlear apex 134. More specifically, the cochlea 140 is generally a conical spiral structure (i.e., the spiral-like shape) and the apical region 138 of the cochlea 140 is generally the last/final (i.e., most apical) 360 degrees of the cochlea and encompasses the cochlea areas tonotopically associated with frequencies below 1000 Hz.

FIG. 1C specifically illustrates the scala tympani 108, relative to the modiolus 112. Shown in FIG. 1C is the modiolar wall 120 (e.g., the wall of the scala tympani 108 adjacent the modiolus 112) and the lateral wall 118 (e.g., the wall of the scala tympani 108 positioned opposite to the modiolus 112, adjacent to the basilar membrane 124). Also shown in FIG. 1C is a cochlea opening 142 which may be, for example, a natural opening (e.g., the round window) or a surgical opening (e.g., cochleostomy).

For individuals who have significant residual hearing, particularly high frequency hearing in the basal region of the cochlea, intra-cochlea drug delivery is challenging without introducing or increasing hearing loss. For example, using conventional arrangements, it is difficult to insert devices into the cochlea without interfering with (e.g., damaging, contacting, etc.), for example, the spiral ligament, the basilar membrane, destroying hair cells, etc., all of which can introduce or increase sensorineural hearing loss. As such, when residual hearing is present, conventional therapeutic substance delivery techniques generally do not deliver the therapeutic substances directly into the cochlea. Instead, the therapeutic substances tend to be delivered outside of the cochlea (e.g., at the outside of the round window or other opening). However, these conventional techniques can, for example, lack efficacy and generally fail to deliver the therapeutic substances at specific locations within the cochlea, as needed (e.g., therapeutic substances delivered via conventional techniques can fail to reach the apical region of the cochlea).

As a result of the above and other reasons, presented herein are therapeutic substance delivery devices configured to deliver therapeutic substances inside the fluidically-sealed cochlea 140 (or other body chamber) without interfering with (e.g., damaging) the hearing structures of the cochlea (e.g., without introducing additional hearing loss), such as the spiral ligament, the stria vascularis, the basilar membrane, the organ of Corti, the cochlear aqueduct, etc. In general, therapeutic substance delivery devices in accordance with certain embodiments presented herein comprise a delivery substrate (substrate) configured to be inserted into a cochlea of a recipient. The delivery substrate is configured to assume a perimodiolar position within the cochlea (e.g., close proximity to the modiolar wall) and is structural arranged (e.g., sized, shaped, etc.) such that implantation of the delivery substrate does not interfere with or otherwise affect lateral structures of the cochlea, including the spiral ligament (at the lateral wall) and, in some anatomy, the stria vascularis, the basilar membrane, the organ of Corti, and/or the cochlear aqueduct. In certain embodiments, the delivery substrate includes one or more therapeutic substance disposed in or on the substrate. In the context of intra-cochlear applications, the therapeutic substance delivery devices are sometimes referred to as “perimodiolar” therapeutic substance delivery devices, which enable drug delivery to the cochlea while preserving acoustic function of the cochlea.

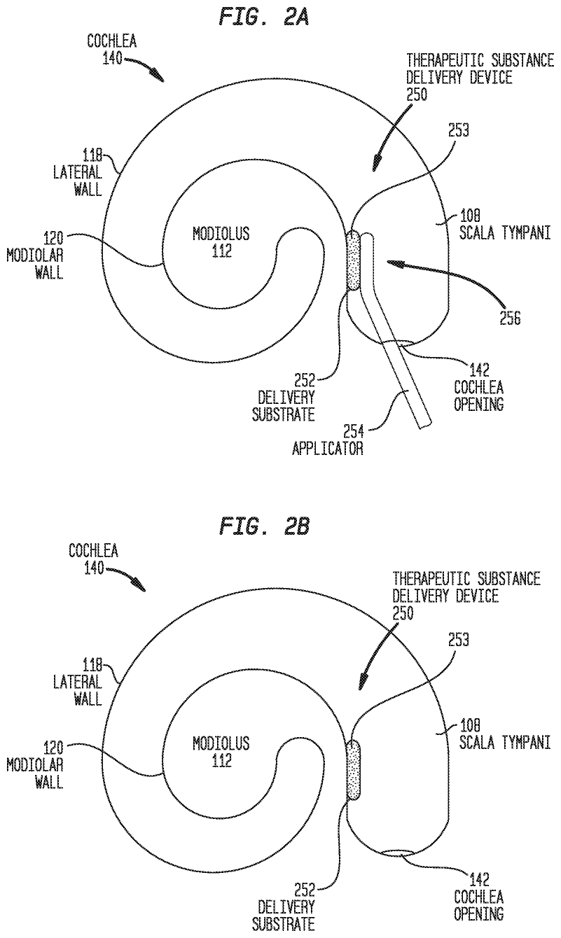

FIGS. 2A-2B, 3, 4A-4C, 5A-5B, and 6 illustrate example therapeutic substance delivery devices, in accordance with various embodiments presented herein. For ease of description, the therapeutic substance delivery devices in each of FIGS. 2A-2B, 3, 4A-4C, 5A-5B and 6 will be described with reference to cochlea 140 of FIGS. 1A-1C.

Referring first to FIGS. 2A and 2B, shown is a first example therapeutic substance delivery device 250. In particular, FIG. 2A illustrates the therapeutic substance delivery device 250 during implantation of the device 250 into the cochlea 140, while FIG. 2B illustrates the therapeutic substance delivery device 250 following (after) implantation of the device 250 into the cochlea 140.

In the example of FIGS. 2A and 2B, the therapeutic substance delivery device 250 comprises a therapeutic substance delivery substrate (delivery substrate) 252 that includes one or more therapeutic substances 253. That is, the delivery substrate 252 is loaded/doped with, and/or coated with, one or more therapeutic substances 253 that are configured to be released into the cochlea 140 via, for example, elution, resorption, etc. The delivery substrate 252 is shown with an associated elongate applicator 254. In this example, the delivery substrate 252 has a general cylindrical or disk shape and is formed from, for example, a silicone or gel material.

As shown in FIG. 2A, the delivery substrate 252 is initially detachably coupled to a distal end 256 of the elongate applicator 254 (e.g., guidewire). The distal end 256 of the elongate applicator 254, with the delivery substrate 252 attached thereto, is inserted through the cochlea opening 142. The elongate applicator 254 is manipulated such that the delivery substrate 252 is placed in contact with the modiolar wall 120 of the cochlea 140. In these examples, the delivery substrate 252 itself has adhesive properties such that, when placed in contact with the modiolar wall 120, the delivery substrate 252 self-adheres to the modiolar wall 120. In other words, in the embodiments of FIGS. 2A and 2B, the delivery substrate 252 is an “adhesive” substrate configured to self-adhere to the modiolar wall 120.

As noted above, the scala tympani 108 is filled with perilymph, meaning the modiolar wall 120 is a fluid-covered surface. As such, the delivery substrate 252 is formed from, or at least includes, a type of adhesive that can adhere to a wet (fluid-covered) surface (e.g., tissue adhesives with strong wet adhesion, stability under physiological conditions, and, for example, rapid curing/cross-linking without excessive heat generation, nontoxicity, cytocompatibility, minimum swelling, modulus comparable to the underlying tissue, biodegradability, and bioresorbability). In certain examples, the delivery substrate 252 be formed from or include nanoparticle adhesives, fibrin glue (fibrin), and polyethylene glycol-based adhesives, Polyvinyl Alcohol (PVA), PEG and PEG-polysaccharide/protein combinations, bioinspired adhesives such as Mussels, barnacles sandcastle worms, caddisflies, and geckos, slug-inspired adhesives, cyanocrylates, GRFG, Polysaccharide based adhesives, Polypetite based adhesives, Protein based adhesives, (Chitosan, Dextran, gelatin, etc.), etc. Hyrdogels that are not adhesive, per se, can have gel like properties to be sufficiently tacky or surface energy (e.g., water bonds, etc., because there are no forces at play in the cochlea). Moreover, various surface topography to achieve adhesion, such as gecko feet, spider feet, Velcro etc. can be used in different embodiments.

As noted, the delivery substrate 252 is configured to adhere to the modiolar wall 120. However, as noted above, the delivery substrate 252 is also initially detachably coupled to the distal end 256 of the elongate applicator 254. In operation, when the delivery substrate 252 adheres to the modiolar wall 120, the delivery substrate 252 also disengages from the distal end 256 of the elongate applicator 254. That is, at least one of the delivery substrate 252 and/or the distal end 256 of the elongate applicator 254 is also configured such that the delivery substrate 252 is released from the distal end 256 and remains in place on the modiolar wall 120 (e.g., mechanical release, release via a weak adhesive coupling, etc.). In this way, the elongate applicator 254 can be removed from the cochlea 140 via the cochlea opening 142. Once the elongate applicator 254 is removed from the cochlea 140, the cochlea opening 142 can be surgically closed (e.g., via sutures, a sealing device, etc.) with the delivery substrate 252 inside the cochlea 140.

In accordance with certain embodiments presented herein, the delivery substrate 252 is fully bioresorbable, meaning the delivery substrate 252 will, over time, be fully resorbed by the body (e.g., the delivery substrate 252 is constructed of a degradable material which may or may not be the therapeutic compound or drug and serves as the scaffold and support for the therapeutic substance). In certain embodiments, the delivery substrate 252 can resorb at a rate that precludes scar formation and/or permanent establishment of fibrosis.

As noted, the delivery substrate 252 has a general disc/cylinder shape. The delivery substrate 252, with the associated applicator 254, are structurally arranged (e.g., sized and shaped) to be inserted into the scala tympani 108 so as not to interfere with acoustic hearing structures in the scala tympani 108, including the spiral ligament membrane 130 located at the lateral wall 118, the organ of Corti 110, the basilar membrane 124, the stria vascularis, etc., both during and after surgery. For example, the delivery substrate 252 can be configured to have a minimum spacing, following implantation, from any or all of the spiral ligament membrane 130, organ of Corti 110, basilar membrane 124, etc. The minimum spacing can be based, for example, on an estimated size of the fibrosis/tissue response/growth and the likelihood of connective tissue becoming an interference with the basilar membrane or the aqueduct. The reason to avoid the spiral ligament area is the same, likely tissue outgrowth up toward the basilar membrane (e.g., the anatomy and geometry of the delivery substrate 252 can be arranged to maximum possible spacing from the organ of corti 110 and/or spiral ligament membrane 130). In certain embodiments, the minimum spacing between the delivery substrate 252 and any tissue other than the modiolar wall 120 is at least around 0.2 mm. In further embodiments, the minimum spacing between the delivery substrate 252 and any tissue other than the modiolar wall 120 is at least around 0.5 mm or greater.

In view of the above, the delivery substrate 252 can have a low aspect ratio (e.g., ratio between height/thickness and width). In certain embodiments, an aspect ratio of at 1:2 or lower, and in further embodiments an aspect ratio of 1:4 or lower, could achieve the desired ratio of therapeutic substance quantity versus “invasiveness”/foreign body size in the cochlea. The dimensions of the scala tympani in the area of interest is in the range of, for example, 1.2 mm-2 mm height (e.g., slightly wider in the lateral dimension) and as such, a maximum “width” of 1 mm and a height of maximum of 300 um may be suitable in certain embodiments. In one specific embodiment, a geometry of 400 um wide by 100 um high may be used. In certain embodiments, the substrate 252 can have a width that is at least twice its associated height (e.g., 20 um×40 um, 100 um×200 um, 100 um×250 um 100 um×300 um, etc.). In further embodiments, the delivery substrate 252 can have a width that is at least four times its height.

Again, the benefit of a low aspect ratio is to keep the delivery substrates 252 away from the critical hearing structures along the lateral wall 118 (e.g., from the spiral ligament membrane 130), where the thickness/height of the substrate (rather than the width) controls the distance/spacing from the lateral wall. A low aspect ratio provides sufficient surface area for therapeutic substance delivery, while providing room such that fibrous growth will not expand to reach the spiral ligament membrane 130 along the lateral wall 118 and/or the basilar membrane 124.

As noted, it is further desirable so as not to interfere or damage acoustic hearing structures in the scala tympani 108 during the insertion/implantation. As such, the applicator 254 is similarly structurally arranged (e.g., sized and shaped) so as to ensure the applicator does not contact, for example, the lateral wall 118 during insertion of the delivery substrate 252. The arrangement of the applicator 254 can vary depending on the arrangement of the delivery substrate 252. In one embodiment, the applicator may have a thickness/height that is at least less than the desired minimum spacing between the delivery substrate 252 and the lateral wall. For example, the applicator 254 could have thickness that is in the range of around 2 mm or less, where round window dimensions could dictate a smaller thickness. That is, the applicator 254 and the delivery substrate 252 generally have a collectively thickness (and width) to fit into the cochlea via the cochlea opening 142.

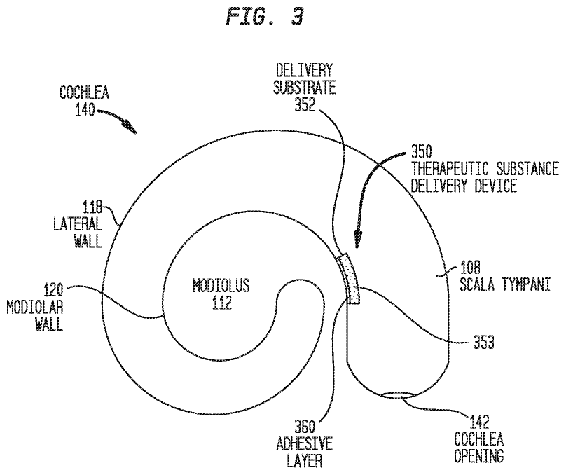

As noted, FIGS. 2A and 2B illustrate an embodiment in which the delivery substrate 252 is formed from a material having adhesive properties that adhere to the modular wall 120 of the cochlea 140. FIG. 3 illustrates an alternative embodiment in which a therapeutic substance delivery device 350 comprises a non-adhesive delivery substrate 352.

More specifically, in the example of FIG. 3, the therapeutic substance delivery device 350 includes a delivery substrate 352 that includes one or more therapeutic substances 353. That is, the delivery substrate 352 is loaded/doped with, and/or coated with, one or more therapeutic substances 353 that are configured to be released into the cochlea 140 via, for example, elution, resorption, etc.

In this example, the delivery substrate 352 has a general cylindrical or disk shape. The delivery substrate 352 can be formed from, for example, a silicone or gel material. Alternatively, the delivery substrate 352 can be formed using a scaffold or crystalline structure where the scaffold/crystal structure is loaded/doped with one or more therapeutic substances 353. In other embodiments, the delivery substrate 352 can be coated with one or more therapeutic substances 353.

Similar to the embodiments of FIGS. 2A and 2B, the therapeutic substance delivery device 350 of FIG. 3 is initially detachably coupled to a distal end of an elongate applicator. The distal end of the elongate applicator, with the therapeutic substance delivery device 350 attached thereto, is inserted through the cochlea opening and elongate applicator is manipulated such that the therapeutic substance delivery device 350 is placed in contact with the modiolar wall 120 of the cochlea 140. However, in the example of FIG. 3, the therapeutic substance delivery device 350 comprises an adhesive 360 positioned on a first surface (modiolar facing surface) 361 of the substrate 352. That is, in contrast to the embodiments of FIGS. 2A and 2B, the delivery substrate 352 may not have significant adhesive properties. However, the adhesive 360 has adhesive properties such that, when placed in contact with the modiolar wall 120, the adhesive 360 adheres the delivery substrate 352 to the modiolar wall 120. In other words, in the embodiments of FIG. 3, the delivery substrate 352 is a “non-adhesive” substrate, but the adhesive 360 adheres the substrate 352 to the modiolar wall 120. FIG. 3 illustrates a specific arrangement in which the adhesive 360 is an adhesive layer 360 attached to the first surface of the delivery substrate 352.

The delivery substrate 352 can be formed from a variety materials, including different biodegradable polymers. For example, the delivery substrate 352 can be formed from, for example, PLGA, Polycaprolactone and copolymers, Polyglycolide, PLA, PLA-PEG copolymers, Polydioxanone, Polytrimethylene carobonate copolymers, Polyanhydrides, Polyorthoesters, Polyphosphazenes, etc.

As noted above, the scala tympani 108 is filled with perilymph, meaning the modiolar wall 120 is a fluid-covered surface. As such, the adhesive layer 360 is formed from, or at least includes, a type of adhesive that can adhere to a fluid-covered surface, as described above with reference to FIGS. 2A and 2B.

As noted, the adhesive layer 360 is configured to adhere to the modiolar wall 120, but the therapeutic substance delivery device 350 is initially detachably coupled to the distal end of an elongate applicator. In operation, when the adhesive layer 360 adheres to the modiolar wall 120, the delivery substrate 352 also disengages from the distal end of the elongate applicator. That is, at least one of the delivery substrate 352 and/or the distal end of the elongate applicator is also configured such that the delivery substrate 352 is released from the distal end and remains in place on the modiolar wall 120. In this way, the elongate applicator can be removed from the cochlea 140 via the cochlea opening 142. Once the elongate applicator is removed from the cochlea 140, the cochlea opening 142 can be surgically closed (e.g., via sutures, a sealing device, etc.) with the therapeutic substance delivery device 350 inside the cochlea 140.

In certain embodiments, the adhesive layer 360 and the delivery substrate 352 are fully bioresorbable, meaning that the entire therapeutic substance delivery device will, over time, be fully resorbed by the body (e.g., the substrate 352 and the adhesive layer 360 are constructed of one or more degradable materials which may or may not be the therapeutic compound or drug and serves as the scaffold and support for the therapeutic substance). In certain embodiments, the delivery substrate 352 and/or the adhesive layer 360 can resorb at a rate that precludes scar formation and/or permanent establishment of fibrosis.

As noted, the delivery substrate 352 has a general disc/cylinder shape. The delivery substrate 352, with the associated applicator 354, are structurally arranged (e.g., sized and shaped) to be inserted into the scala tympani 108 so as not to interfere with acoustic hearing structures in the scala tympani 108, including the spiral ligament membrane 130 located at the lateral wall 118, the organ of Corti 110, the basilar membrane 124, the stria vascularis, etc., both during and after surgery. For example, the delivery substrate 352 can be configured to have a minimum spacing, following implantation, from any or all of the spiral ligament membrane 130, organ of Corti 110, basilar membrane 124, etc. The minimum spacing can be based, for example, on an estimated size of the fibrosis/tissue response/growth and the likelihood of connective tissue becoming an interference with the basilar membrane or the aqueduct. The reason to avoid the spiral ligament area is the same, likely tissue outgrowth up toward the basilar membrane (e.g., the anatomy and geometry of the delivery substrate 352 can be arranged to maximum possible spacing from the organ of corti 110 and/or spiral ligament membrane 130). In certain embodiments, the minimum spacing between the delivery substrate 352 and any tissue other than the modiolar wall 120 is at least around 0.2 mm. In further embodiments, the minimum spacing between the delivery substrate 352 and any tissue other than the modiolar wall 120 is at least around 0.5 mm or greater.

In view of the above, the delivery substrate 352 can have a low aspect ratio (e.g., ratio between height/thickness and width). In certain embodiments, an aspect ratio of at 1:2 or lower, and in further embodiments an aspect ratio of 1:4 or lower, could achieve the desired ratio of therapeutic substance quantity versus “invasiveness”/foreign body size in the cochlea. The dimensions of the scala tympani in the area of interest is in the range of, for example, 1.2 mm-2 mm height (e.g., slightly wider in the lateral dimension) and as such, a maximum “width” of 1 mm and a height of maximum of 300 um may be suitable in certain embodiments. In one specific embodiment, a geometry of 400 um wide by 100 um high may be used. In certain embodiments, the substrate 352 can have a width that is at least twice its associated height (e.g., 20 um×40 um, 100 um×200 um, 100 um×250 um 100 um×300 um, etc.). In further embodiments, the delivery substrate 352 can have a width that is at least four times its height.

Again, the benefit of a low aspect ratio is to keep the delivery substrates 352 away from the critical hearing structures along the lateral wall 118 (e.g., from the spiral ligament membrane 130), where the thickness/height of the substrate (rather than the width) controls the distance/spacing from the lateral wall. A low aspect ratio provides sufficient surface area for therapeutic substance delivery, while providing room such that fibrous growth will not expand to reach the spiral ligament membrane 130 along the lateral wall 118 and/or the basilar membrane 124.

As noted, it is further desirable so as not to interfere or damage acoustic hearing structures in the scala tympani 108 during the insertion/implantation. As such, the applicator 354 is similarly structurally arranged (e.g., sized and shaped) so as to ensure the applicator does not contact, for example, the lateral wall 118 during insertion of the delivery substrate 352. The arrangement of the applicator 354 can vary depending on the arrangement of the delivery substrate 352. In one embodiment, the applicator may have a thickness/height that is at least less than the desired minimum spacing between the delivery substrate 352 and the lateral wall. For example, the applicator 354 could have thickness that is in the range of around 2 mm or less, where round window dimensions could dictate a smaller thickness. That is, the applicator 354 and the delivery substrate 352 generally have a collectively thickness (and width) to fit into the cochlea via the cochlea opening 142.

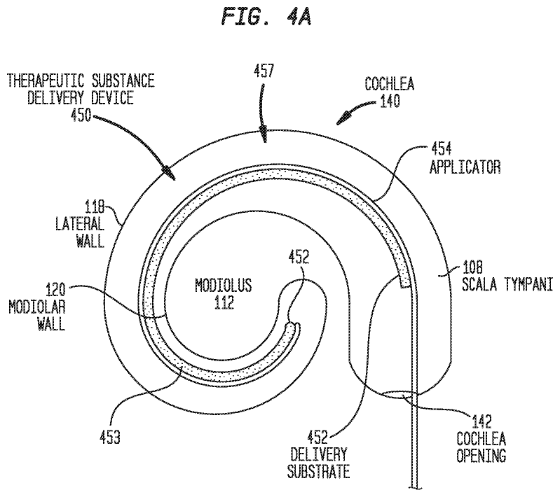

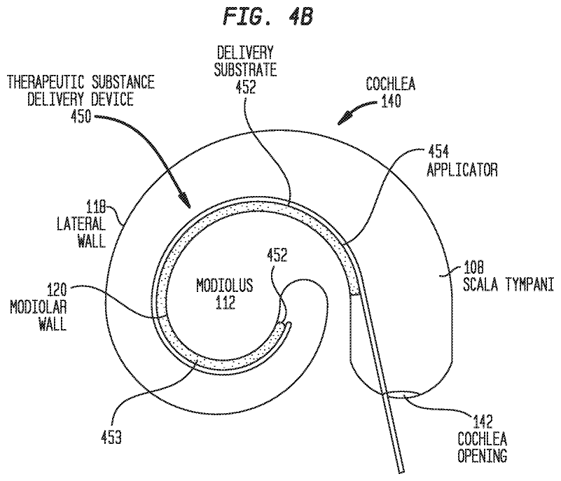

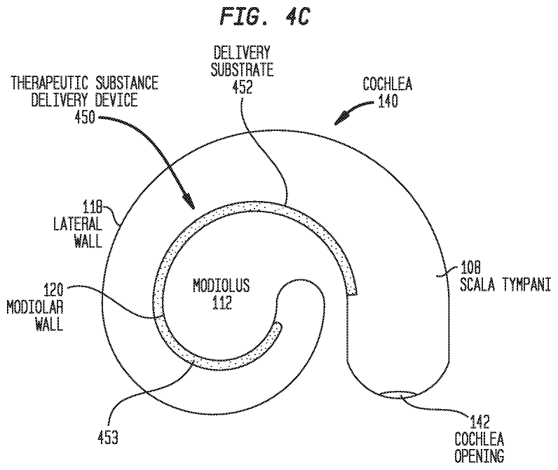

FIGS. 4A, 4B, 4C illustrate another therapeutic substance delivery device 450, in accordance with certain embodiments presented herein. In particular, FIGS. 4A and 4B illustrate the therapeutic substance delivery device 450 during implantation of the device 450 into the cochlea 140, while FIG. 4C illustrates the therapeutic substance delivery device 450 following (after) implantation of the therapeutic substance delivery device 450 into the cochlea 140.

In the example of FIGS. 4A and 4B, the therapeutic substance delivery device 450 comprises an elongate delivery substrate 452 that includes one or more therapeutic substances 453. That is, the delivery substrate 452 is loaded/doped with, and/or coated with, one or more therapeutic substances 453 that are configured to be released into the cochlea 140 via, for example, elution, resorption, etc.

The delivery substrate 452 is shown with an associated elongate applicator 454. In this example, the delivery substrate 452 has an elongate (e.g., tubular, rectangular) shape. The delivery substrate 452 can be formed from, for example, a silicone or gel material. Alternatively, the delivery substrate 452 can be formed using a scaffold or crystalline structure where the scaffold/crystal structure is loaded/doped with one or more therapeutic substances 453. In other embodiments, the delivery substrate 452 can be coated with one or more therapeutic substances 453.

As shown in FIG. 4A, the delivery substrate 452 is initially detachably coupled to an elongate length 457 of the elongate applicator 454. The elongate applicator 454, with the delivery substrate 452 attached thereto, is inserted through the cochlea opening 142. The elongate applicator 454 is manipulated such that the delivery substrate 452 is placed in contact with the modiolar wall 120 of the cochlea 140.

Initially, the delivery substrate 452 and the elongate applicator 454 have a generally straight configuration to facilitate insertion through the cochlea opening 142. As shown in FIGS. 4A and 4B, during insertion into the cochlea 140, the elongate applicator 454 and the delivery substrate 452 are configured to bend/conform to the spiral shape of the scala tympani 108 and adopt a perimodiolar position in the cochlea.

More specifically, in one example, the elongate applicator 454 is pre-curved and is initially held straight. The elongate applicator 454 could be held straight, for example, via a stiffening element (e.g., stylet, sheath, etc.), via a temperature state, etc. The delivery substrate 452 is disposed on the elongate applicator 454, but is resiliently flexible. As shown in FIG. 4A, once it is inserted into the cochlea 140, the elongate applicator 454 returns its pre-curved shape (e.g., via a temperature chance induced by the body, a result of presence in the perilymph, removal of the stiffening element, etc.). The resiliently flexible delivery substrate 452 bends with the elongate applicator 454. The elongate applicator 454 can be activated and the delivery substrate 452 is gently pressed into contact with the modiolar wall 120 of the scala tympani 108, released from the elongate applicator 454, and the delivery substrate 452 adheres there to. The elongate applicator 454 is withdrawn, with minimal disruption to the cochlea 140 (e.g., slides along the delivery substrate 452).

FIG. 4C illustrates the final implanted perimodiolar position/placement for the delivery substrate 452 with the elongate applicator 454 attached thereto, while FIG. 4C illustrates the final implanted perimodiolar position for the delivery substrate 452 with the elongate applicator 454 removed from the cochlea 140. FIG. 4B illustrates the delivery substrate 452 and the elongate applicator 454 during the transition from the initial straight configuration to the final perimodiolar position.

In accordance with certain embodiments, the delivery substrate 452 can, similar to the embodiments of FIGS. 2A and 2B, include adhesive properties. In such embodiments, when the delivery substrate 452 is placed in contact with the modiolar wall 120, the delivery substrate 452 self-adheres to the modiolar wall 120 (e.g., the delivery substrate 452 can be an adhesive substrate). In accordance with alternative embodiments, the delivery substrate 452 can, similar to the embodiments of FIG. 3, have an adhesive layer attached thereto that is configured to adhere the delivery substrate to the modiolar wall 120 (e.g., the delivery substrate 452 can be a non-adhesive substrate, but an adhesive layer adheres the substrate 452 to the modiolar wall 120).

As noted above, the scala tympani 108 is filled with perilymph, meaning the modiolar wall 120 is a fluid-covered surface. As such, in the embodiments of FIGS. 4A and 4B either the delivery substrate 452 (in the case of an adhesive substrate) of the adhesive layer (in the case of a non-adhesive substrate) is formed from, or at least includes, a type of adhesive that can adhere to a fluid-covered surface, as described above with reference to FIGS. 2A, 2B, and 3.

As noted, the therapeutic substance delivery device 450 is configured to adhere to the modiolar wall 120. However, as noted above, the therapeutic substance delivery device 450 is also initially detachably coupled to the elongate length 457 of the elongate applicator 454. In operation, when the therapeutic substance delivery device 450 adheres to the modiolar wall 120, the delivery substrate 452 also disengages from the elongate applicator 454. That is, at least one of the delivery substrate 452 and/or the elongate applicator 454 is also configured such that the delivery substrate 452 is released from the elongate length 457 and remains in place on the modiolar wall 120. In this way, the elongate applicator 454 can be removed from the cochlea 140 via the cochlea opening 142. Once the elongate applicator 454 is removed from the cochlea 140, the cochlea opening 142 can be surgically closed (e.g., via sutures, a sealing device, etc.) with the delivery substrate 452 inside the cochlea 140.

In accordance with certain embodiments presented herein, the delivery substrate 452 is fully bioresorbable, meaning the delivery substrate 452 will, over time, be fully resorbed by the body (e.g., the delivery substrate 452 is constructed of a degradable material which may or may not be the therapeutic compound or drug and serves as the scaffold and support for the therapeutic substance). In certain embodiments, the delivery substrate 452 can resorb at a rate that precludes scar formation and/or permanent establishment of fibrosis.

FIGS. 4A-4C have been generally described with the use of some form of adhesive material to attach the delivery substrate 452 to the modiolar wall 120. In alternative embodiments of FIGS. 4A-4C, no adhesive material is provided and natural forces of attraction/adhesion between the delivery substrate 452 material (e.g., polymer) and the tissue of the modiolar wall 120 are sufficient to retain the therapeutic substance delivery device 450 in the perimodiolar position.

As noted, the delivery substrate 452, with the associated applicator 454, are structurally arranged (e.g., sized and shaped) to be inserted into the scala tympani 108 so as not to interfere with acoustic hearing structures in the scala tympani 108, including the spiral ligament membrane 130 located at the lateral wall 118, the organ of Corti 110, the basilar membrane 124, the stria vascularis, etc., both during and after surgery. As such, a perimodiolar position along the modiolar wall 120 (e.g., the wall directly opposite the lateral wall 118), as described above, serves to substantially eliminate or reduce the impact the therapeutic substance delivery device 450 on these hearing structures. However, impact on the hearing structures along the lateral wall 118 can also be reduced via proper relative sizing of the delivery substrate 452 to the section/region of the cochlea 140 into which the delivery substrate 452 is inserted.

The delivery substrate 452 can be configured to have a minimum spacing, following implantation, from any or all of the spiral ligament membrane 130, organ of Corti 110, basilar membrane 124, etc. The minimum spacing can be based, for example, on an estimated size of the fibrosis/tissue response/growth and the likelihood of connective tissue becoming an interference with the basilar membrane or the aqueduct. The reason to avoid the spiral ligament area is the same, likely tissue outgrowth up toward the basilar membrane (e.g., the anatomy and geometry of the delivery substrate 452 can be arranged to maximum possible spacing from the organ of corti 110 and/or spiral ligament membrane 130). In certain embodiments, the minimum spacing between the delivery substrate 452 and any tissue other than the modiolar wall 120 is at least around 0.2 mm. In further embodiments, the minimum spacing between the delivery substrate 452 and any tissue other than the modiolar wall 120 is at least around 0.5 mm or greater.

As noted, the delivery substrate 452 has an elongate (e.g., tubular, rectangular) shape. In general, the delivery substrate 452 has a low aspect ratio (e.g., ratio between height/thickness and width or, for example, 1:2 or lower, 1:4 or lower, etc.). In certain embodiments, the delivery substrate 452 has a width that is at least twice its height (e.g., 20 um×40 um, 100 um×200 um, 100 um×250 um, 100 um×300 um, etc.). In further embodiments, the delivery substrate 452 has a width that is at least four times its height. Again, the benefit of a low aspect ratio is to keep the delivery substrate 452 away from the critical hearing structures (e.g., along the lateral wall 118) where the thickness/height of the substrate (rather than the width) controls the distance/spacing from the lateral wall. A low aspect ratio provides sufficient surface area for therapeutic substance delivery, while providing room such that fibrous growth will not expand to reach the hearing structures along the lateral wall 118.

As noted, it is further desirable so as not to interfere or damage acoustic hearing structures in the scala tympani 108 during the insertion/implantation. As such, the applicator 454 is similarly structurally arranged (e.g., sized and shaped) so as to ensure the applicator does not contact, for example, the lateral wall 118 during insertion of the delivery substrate 452. The arrangement of the applicator 454 can vary depending on the arrangement of the delivery substrate 452. In one embodiment, the applicator 454 may have a thickness/height that is at least less than the desired minimum spacing between the delivery substrate 452 and the lateral wall. For example, the applicator 454 could have thickness that is in the range of around 2 mm or less, where round window dimensions could dictate a smaller thickness. That is, the applicator 454 and the delivery substrate 452 generally have a collectively thickness (and width) to fit into the cochlea via the cochlea opening 142.

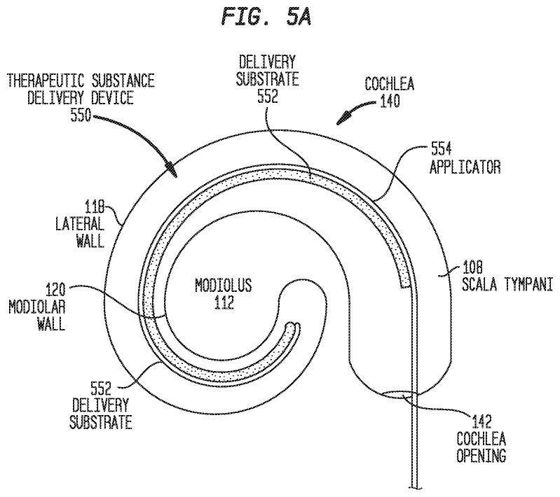

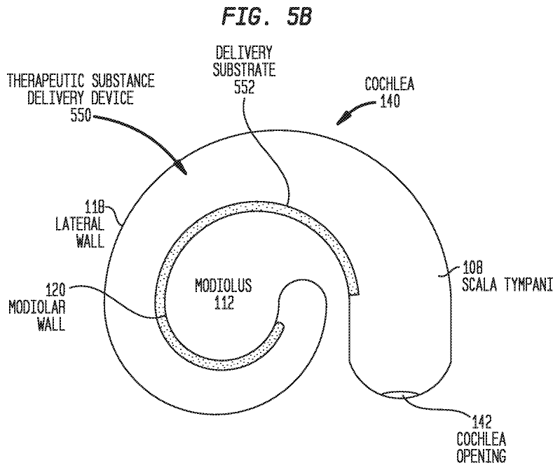

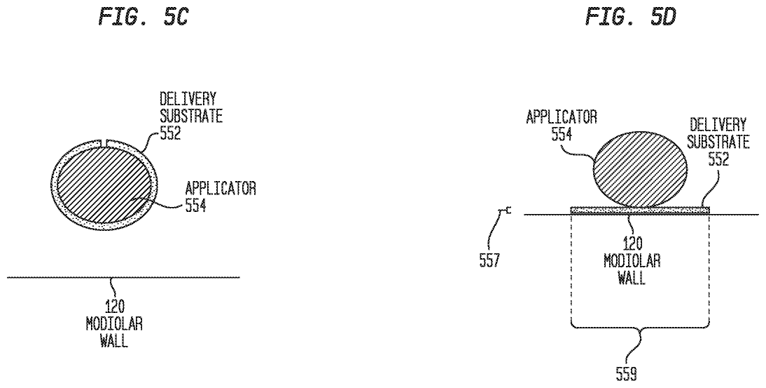

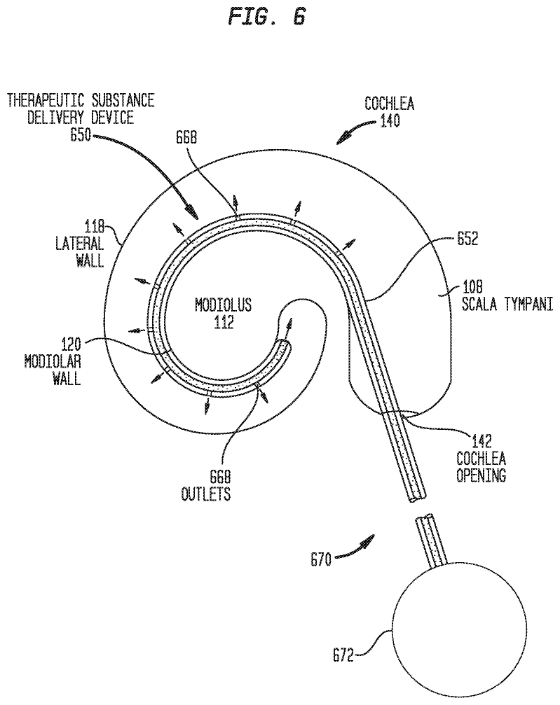

FIGS. 5A-5D illustrate another therapeutic substance delivery device 550, in accordance with certain embodiments presented herein. In particular, FIG. 5A illustrates the therapeutic substance delivery device 550 during implantation of the device 550 into the cochlea 140, while FIG. 5B illustrates the therapeutic substance delivery device 550 following (after) implantation of the therapeutic substance delivery device 550 into the cochlea 140. In addition, FIG. 5C is cross-sectional view of the therapeutic substance delivery device 550 during insertion into the cochlea 140, while FIG. 5D is a cross-sectional view of the therapeutic substance delivery device 550 following implantation of the delivery device 550 into the cochlea 140. For ease of illustration, all of the cochlea 140 except for the modiolar wall 120 has been omitted from FIGS. 5C and 5D.

In the example of FIGS. 5A-5D, the therapeutic substance delivery device 550 again comprises a delivery substrate 552 that is loaded/doped with, and/or coated with, one or more therapeutic substances 533. However, in these examples, the delivery substrate 552 is a thin sheet of paper-like material (e.g., cigarette paper material) that is, as shown in FIG. 5C, initially wrapped around an elongate applicator 554. As such, the delivery substrate 552 of FIGS. 5A-5D is sometimes referred to herein as a “paper deliver substrate” or simply “paper substrate.”

Initially, the elongate applicator 554 have a generally straight configuration to facilitate insertion through the cochlea opening 142. As shown in FIG. 5A, during insertion into the cochlea 140, the elongate applicator 554 (with the paper delivery substrate 552 disposed there around) is configured to bend/conform to the spiral shape of the scala tympani 108 and adopt a perimodiolar position in the cochlea. That is, the elongate applicator 554, with the paper delivery substrate 552 disposed there around, is inserted through the cochlea opening 142. Subsequently, the elongate applicator 554 bends (e.g., as described above) and is manipulated such that the paper delivery substrate 552 is placed in contact with the modiolar wall 120 of the cochlea 140. The contact between the paper delivery substrate 552 and the modiolar wall, in the fluid-filled cochlea 140, causes the paper delivery substrate 552 to release from the elongate applicator 554 and adopt a generally flat configuration along the modiolar wall 120 (as shown in FIG. 5D). Stated differently, the paper delivery substrate 552 is configured to release from the elongate applicator 554 and spring gently into a perimodiolar position, forming a “wet” bond with the surface of the modiolar wall 120 (e.g., the paper bonds to the wet surface and remains adhered thereto).

FIG. 5D illustrates the final perimodiolar position/placement for the delivery substrate 552 with the elongate applicator 554 adjacent thereto, while FIG. 5B illustrates the final implanted perimodiolar position for the delivery substrate 552 with the elongate applicator 554 removed from the cochlea 140. Similar to the above embodiments, once the elongate applicator 554 is removed from the cochlea 140, the cochlea opening 142 can be surgically closed (e.g., via sutures, a sealing device, etc.) with the delivery substrate 552 inside the cochlea 140.

In accordance with certain embodiments presented herein, the delivery substrate 552 is fully bioresorbable, meaning the delivery substrate 552 will, over time, be fully resorbed by the body (e.g., the delivery substrate 552 is constructed of a degradable material which may or may not be the therapeutic compound or drug and serves as the scaffold and support for the therapeutic substance). In certain embodiments, the delivery substrate 552 can resorb at a rate that precludes scar formation and/or permanent establishment of fibrosis.

As noted, the paper delivery substrate 552 has an elongate (e.g., tubular, rectangular) shape. However, since the paper delivery substrate 552 is formed from a paper-like material, the delivery substrate 452 has an ultra-low aspect ratio (e.g., ratio between height/thickness and width). In certain embodiments, the delivery substrate 552 has a deployed/final height/thickness 557 and a width 559. In certain embodiments, the maximum width could be slightly greater than the width of the chamber (e.g., greater than 1 mm for the cochlea) as the paper unwraps and sits right into the modiolar wall. As such, in the context of the scala tympani, a maximum width of 1.8 mm and a thickness/height of about 250 um or lower maybe used. In one embodiment, the delivery substrate 452 has a width of about, or greater than, 1 mm and a thickness/height of about or less than 50 um. The applicator 454 could have thickness/diameter that is in the range of around 2 mm or less, where round window dimensions could dictate a smaller thickness.

As noted, FIGS. 2A-2B, 3, 4A-4C, and 5A-5D illustrate embodiments in which a delivery substrate of a therapeutic substance delivery device is loaded/doped with, and/or coated with, one or more therapeutic substances. In accordance with alternative embodiments, a delivery substrate in accordance with embodiments presented herein can include an integrated cannula for delivery of one or more therapeutic substances from a source located outside of the cochlea 140 (or other body chamber). An example of one such arrangement is shown in FIG. 6.

More specifically, FIG. 6 illustrates a therapeutic substance delivery device 650 following (after) implantation of the therapeutic substance delivery device 650 into the cochlea 140. In the example of FIG. 6, the therapeutic substance delivery device 650 comprises an elongate delivery substrate 652 with an elongate cannula 664 extending longitudinally through the substrate. In addition, the delivery substrate 652 includes one or more outlets/ports 668 fluidically coupling the cannula 664 to an exterior surface 665 of the delivery substrate 652.

Initially, the delivery substrate 652 and has a generally straight configuration to facilitate insertion through the cochlea opening 142. However, as shown in FIG. 6, during insertion into the cochlea 140, the delivery substrate 652 is configured to bend/conform to the spiral shape of the scala tympani 108 and adopt a perimodiolar position in the cochlea.

More specifically, in one example, delivery substrate 652 is pre-curved and is initially held straight, for example, via a stiffening element (e.g., guidewire, stylet, sheath, etc.), via a temperature state, etc. As shown in FIG. 6, once it is inserted into the cochlea 140, the delivery substrate 652 returns to the pre-curved shape (e.g., via a temperature chance induced by the body, a result of presence in the perilymph, removal of the stiffening element, etc.). As it bends or thereafter, the delivery substrate 652 is gently pressed into contact with the modiolar wall 120 of the scala tympani 108, and the delivery substrate 552 adheres there to.

As shown, a proximal end 670 of the cannula 664 is configured to receive one or more therapeutic substances, in a liquid/fluid form, from a therapeutic substance source 672 located outside (external to) the cochlea 140. For example, in certain embodiments, the proximal end 670 of the cannula 664 is fluidically coupled to a refillable or non-refillable reservoir that is, for example, implanted in the body of the recipient, a pump mechanism, etc. Alternatively, the proximal end 670 of the cannula 664 terminates at a self-healing septum that is configured to receive repeated injections using a needle and syringe. These example therapeutic substance sources (e.g., a reservoir, a pump, a self-healing septum) are merely illustrative and that the proximal end 670 of the cannula 664 could be coupled to other types of therapeutic substance sources.

In accordance with certain embodiments, the delivery substrate 652 is fully bioresorbable, meaning the delivery substrate 652 will, over time, be fully resorbed by the body (e.g., the delivery substrate 652 is constructed of a degradable material which may or may not be the therapeutic compound or drug and serves as the scaffold and support for the therapeutic substance). In certain embodiments, the delivery substrate 652 can resorb at a rate that precludes scar formation and/or permanent establishment of fibrosis.

As noted, the delivery substrate 652 is structurally arranged (e.g., sized and shaped) to be inserted into the scala tympani 108 so as not to interfere with acoustic hearing structures in the scala tympani 108, including the spiral ligament membrane 130 located at the lateral wall 118, the organ of Corti 110, the basilar membrane 124, the stria vascularis, etc., both during and after surgery. As such, a perimodiolar position along the modiolar wall 120 (e.g., the wall directly opposite the lateral wall 118), as described above, serves to substantially eliminate or reduce the impact the therapeutic substance delivery device 652 on these hearing structures. However, impact on the hearing structures along the lateral wall 118 can also be reduced via proper relative sizing of the delivery substrate 652 to the section/region of the cochlea 140 into which the delivery substrate 652 is inserted.

The delivery substrate 652 can be configured to have a minimum spacing, following implantation, from any or all of the spiral ligament membrane 130, organ of Corti 110, basilar membrane 124, etc. The minimum spacing can be based, for example, on an estimated size of the fibrosis/tissue response/growth and the likelihood of connective tissue becoming an interference with the basilar membrane or the aqueduct. The reason to avoid the spiral ligament area is the same, likely tissue outgrowth up toward the basilar membrane (e.g., the anatomy and geometry of the delivery substrate 652 can be arranged to maximum possible spacing from the organ of corti 110 and/or spiral ligament membrane 130). In certain embodiments, the minimum spacing between the delivery substrate 652 and any tissue other than the modiolar wall 120 is at least around 0.2 mm. In further embodiments, the minimum spacing between the delivery substrate 652 and any tissue other than the modiolar wall 120 is at least around 0.5 mm or greater.

As noted, the delivery substrate 652 has an elongate (e.g., tubular, rectangular) shape with an elongate cannula 664 extending thereto. In general, the delivery substrate 652 has a low aspect ratio (e.g., ratio between height/thickness and width). In certain embodiments, the delivery substrate 652 has a width that is at least twice its height (e.g., 20 um×40 um, 100 um×200 um, 100 um×250 um, 100 um×300 um, etc.). In further embodiments, the delivery substrate 652 has a width that is at least four times its height. In one embodiment, the delivery substrate 652 could have an aspect ratio of one (1), for a circular cannula, could be used. However, it is to be appreciated that other dimensions and/or other aspect ratios are possible.

Again, the benefit of a low aspect ratio is to keep the delivery substrate 652 away from the critical hearing structures along the lateral wall 118, where the thickness/height of the substrate (rather than the width) controls the distance/spacing from the lateral wall. A low aspect ratio provides sufficient surface area for therapeutic substance delivery, while providing room such that fibrous growth will not expand to reach the hearing structures along the lateral wall 118.

Delivery substrates in accordance with embodiments presented herein can have a number of different structural arrangements. For example, as shown in FIGS. 2A-2C, the delivery substrates can have a generally solid structure from which therapeutic substances can be delivered into a body chamber. For example, the delivery substrates can formed from a solid material, such as a gel or silicone that is configured to elute one or more therapeutic substances.

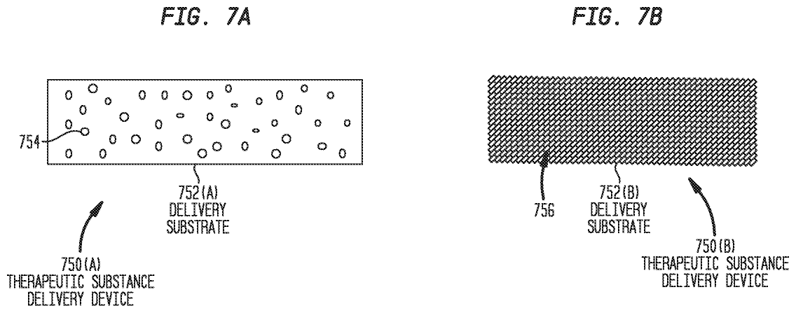

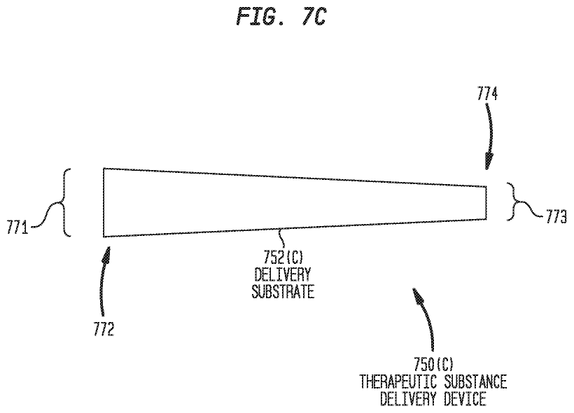

FIG. 7A is a side-view of a portion of an alternative therapeutic substance delivery device 750(A) comprising a delivery substrate 752(A). In this example, the delivery substrate 752(A) has a porous structure (e.g., the delivery substrate 752(A) includes a plurality of pores 754 therein. Similarly, FIG. 7B is a side-view of a portion of an alternative therapeutic substance delivery device 750(B) comprising a delivery substrate 752(B). In this example, the delivery substrate 752(B) has a lattice structure 756. The embodiments of FIGS. 7A and 7B can be advantageous over a solid structure in that these arrangements provide increased surface area per unit volume for elution and can enable more control over the elution/absorption rate. That is, the embodiments of FIGS. 7A and 7B provide a greater surface area, which in turn can be tailored to provide a predetermined delivery profile (e.g., delivery time), a better cellular response relative to a solid substrate, etc.

Therapeutic substance delivery devices presented herein can also have a variable aspect ratio along the elongate lengths thereof facilitating deeper insertion of the therapeutic substance delivery devices into a body chamber. For example, FIG. 7C illustrates a top-view of a portion of an alternative therapeutic substance delivery device 750(C) comprising a delivery substrate 752(C). In this example, the delivery substrate 752(C) has a first width (lateral dimension) 771 at a proximal end 772 of the delivery substrate 752(C), and a second width 773 at a distal end 774 of the delivery substrate 752(C). The second width 773 is less than the first width 771. As such, in this example, the delivery substrate 752(C) has a variable (e.g., distally-decreasing) width. In certain embodiments, the thickness of the delivery substrate 752(C) may be substantially the same across the elongate length of the substrate, meaning that the delivery substrate 752(C) has a variable (e.g., distally-decreasing) aspect ratio. In other embodiments, the thickness of the delivery substrate 752(C) may also be variable (e.g., distally-decreasing). In such embodiments, the aspect ratio of the delivery substrate 752(C) may remain substantially constant (e.g., the thickness changes in proportion to the width) or can be variable.

In any of the above embodiments, a delivery substrate can include (e.g., have disposed therein or have disposed thereon) one or more therapeutic substances. In certain embodiments, a delivery substrate can include a plurality of therapeutic substances. For example, different portions of a delivery substrate can include different therapeutic substances or a single portion of a delivery substrate can include different therapeutic substances. In such embodiments, the different therapeutic substances can have different therapeutic effects, different release profiles (e.g., different release timelines), and/or other differences. For example, by incorporating degrading protrusions which are coated in, or constructed of, different therapeutic substances, it is possible to produce a combination of burst and sustained release profiles.

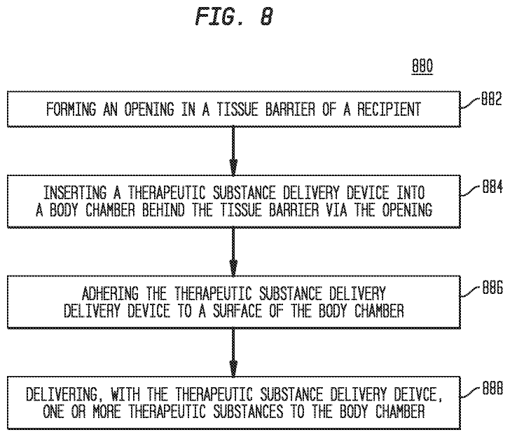

FIG. 8 is a flowchart illustrating an example method 880, in accordance with embodiments presented herein. Method 880 begins at 882 where a surgeon forms an opening in a tissue barrier of a recipient. At 884, the surgeon inserts a therapeutic substance delivery device into a body chamber behind the tissue barrier via the opening. At 886, the surgeon adheres the therapeutic substance delivery device to a surface of the body chamber. At 888, the therapeutic substance delivery device delivers one or more therapeutic substances into the body chamber.

As described elsewhere herein, the tissue barrier may comprise, for example, a tissue barrier associated with a recipient's cochlea. The opening in the tissue barrier can be the round window (round window membrane), the oval window (oval window membrane), a cochleostomy in the bony wall of the cochlea, etc. The body chamber can be a fluidically-sealed chamber, such as the scala tympani, scala media, scala vestibuli, semi-circular canals, any other volume of the labyrinthine, etc.

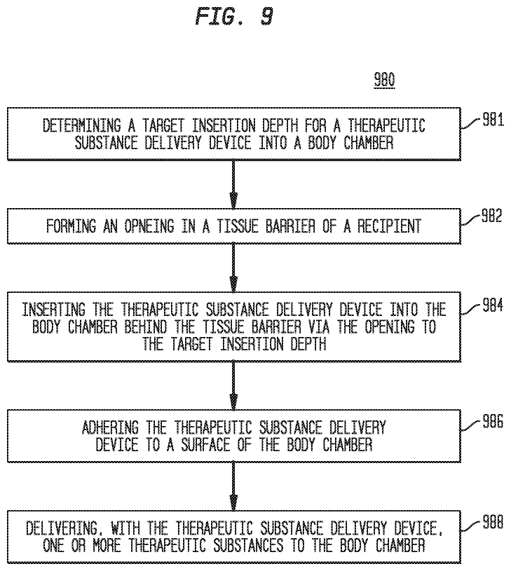

FIG. 9 is a flowchart illustrating an example method 980, in accordance with embodiments presented herein. Method 980 is similar to method 880 of FIG. 9, but further includes the step of, determining a target/selected insertion (e.g., angular insertion depth) for the therapeutic substance delivery device within the body chamber, prior to implanting the therapeutic substance delivery device. This is shown in FIG. 9 at 981.

For an elongate therapeutic substance delivery device, the target insertion depth can comprise a target depth (e.g., maximum depth, minimum depth, etc.) for a distal end of the therapeutic substance delivery device. For a non-elongate therapeutic substance delivery device (e.g., a disk), the target insertion depth can comprise a target depth at which the non-elongate therapeutic substance delivery device should be centered.

In certain embodiments, the target insertion depth can be determined based on pharmacokinetics modelling, with the therapeutic substances targeting a particular region of the body chamber (e.g., cochlea). In certain embodiments, the target insertion depth can be determined based on the Greenwood function describing the tonotopic arrangement of the cochlea (e.g., how the frequency varies in the cochlea). For example, the Greenwood function can be used to determine a target tonotopic frequency location within the cochlea for delivery of therapeutic substances thereto. In such example, the therapeutic substance delivery device could be implanted (e.g., adhered to the modiolar wall) such that the device delivers the one or more therapeutic substances to the target tonotopic frequency location.

After determining a target placement (location/position) for the therapeutic substance delivery device, at 981, a surgeon forms an opening in a tissue barrier of a recipient. At 884, the surgeon inserts a therapeutic substance delivery device into a body chamber behind the tissue barrier via the opening to the target placement. At 986, the surgeon adheres the therapeutic substance delivery device to a surface of the body chamber. At 988, the therapeutic substance delivery device delivers one or more therapeutic substances into the body chamber.

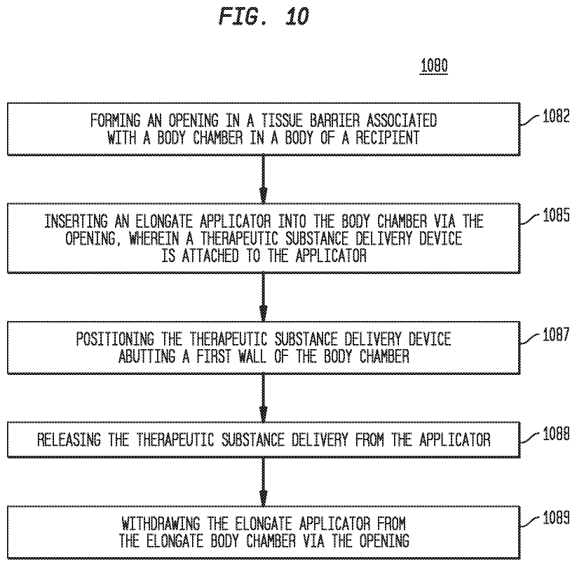

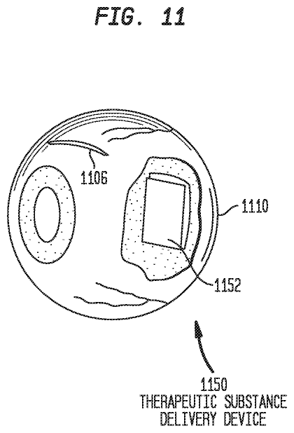

FIG. 10 is a flowchart illustrating an example method 1080, in accordance with embodiments presented herein. Method 1080 begins at 1082 where a surgeon forms an opening in a tissue barrier associated with a body chamber in a body of a recipient. At 1085, the surgeon inserts an elongate applicator into the body chamber via the opening, wherein a therapeutic substance delivery device is attached to the applicator. At 1087, the surgeon positions the therapeutic substance delivery device abutting a first wall of the body chamber. At 1088, the surgeon releases the therapeutic substance delivery from the applicator. At 1089, the surgeon withdraws the elongate applicator from the elongate body chamber via the opening.

As described elsewhere herein, therapeutic substance delivery devices in accordance with certain embodiments presented herein can be used to deliver therapeutic substances to a number of different body chambers located, for example, behind a number of different tissue barriers, including, fluidically-sealed body chambers located behind the blood-brain barrier (BBB), behind the blood-labyrinth barrier (BLB), behind the blood-ocular barrier (BOB), which includes the blood-aqueous barrier (BAB) and the blood-retinal barrier (BRB), and so on. For example, therapeutic substance delivery devices in accordance with certain embodiments presented herein can be used to deliver therapeutic substances the scala tympani, the scala media, the scala vestibuli, the semi-circular canals, any other volume of the labyrinthine, the retina, etc.

FIG. 11 depicts an exemplary embodiment of a therapeutic substance delivery device 1150 for use in the retina of a recipient. In the example of FIG. 11, the therapeutic substance delivery device 1150 includes a delivery substrate 1152 configured to be positioned proximate the retina 1110. The therapeutic substance delivery device 1150 is inserted through a surgical incision 1106 through the eye wall and is configured to be adhered to a surface proximate the retina 1110.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners. For example, the embodiments of FIGS. 2A-2B could be combined with any of the embodiments of FIG. 3, 4A, 4B, 4C, 5A, 5B, 6, 7A, 7B, 7C, 8, or 9. Similarly, the embodiments of FIG. 3 could be combined with any of the embodiments of FIG. 4A, 4B, 4C, 5A, 5B, 6, 7A, 7B, 7C, 8, or 9. Similarly, the embodiments of FIGS. 4A, 4B, and/or 4C could be combined with any of the embodiments of FIG. 5A, 5B, 6, 7A, 7B, 7C, 8, or 9. Similarly, the embodiments of FIGS. 5A and/or 5B could be combined with any of the embodiments of FIG. 6, 7A, 7B, 7C, 8, or 9. Similarly, the embodiments of FIG. 6 could be combined with any of the embodiments of FIG. 7A, 7B, 7C, 8, or 9. Similarly, the embodiments of FIGS. 7A, 7B, and/or 7C could be combined with any of the embodiments of FIG. 8 or 9. Similarly, the embodiments of FIG. 8 could be combined with any of the embodiments of FIG. 9.

1. A method, comprising:
inserting a therapeutic substance delivery device into a body chamber of a recipient;
adhering the therapeutic substance delivery device to a surface of the body chamber; and
delivering, with the therapeutic substance delivery device, one or more therapeutic substances to the body chamber.

2. (canceled)

3. (canceled)

4. (canceled)

5. The method of claim 1, wherein adhering the therapeutic substance delivery device to the surface of the body chamber comprises:
adhering the therapeutic substance delivery device to the surface of the body chamber via a wet bond formed between the therapeutic substance delivery device and the surface of the body chamber.

6. The method of claim 1, wherein the body chamber is a cochlea canal of the recipient, and wherein adhering the therapeutic substance delivery device to a surface of the body chamber comprises:
adhering the therapeutic substance delivery device to a modiolar wall of the cochlea canal.

7. The method of claim 6, further comprising:
determining a target insertion depth for the therapeutic substance delivery device within the cochlea; and
inserting the therapeutic substance delivery device to the target insertion depth.

8. The method of claim 7, wherein determining a target insertion depth for the therapeutic substance delivery device within the cochlea comprises:
determining a target tonotopic frequency location within the cochlea, and wherein the method further comprises:
adhering the therapeutic substance delivery device to a location within the cochlea to deliver one or more therapeutic substances to the target tonotopic frequency location.

9. The method of claim 7, wherein determining a target insertion depth for the therapeutic substance delivery device within the cochlea comprises:
determining the target insertion depth based on pharmacokinetics modelling, with the therapeutic substances targeting a particular region of the cochlea.

10. The method of claim 1, wherein the therapeutic substance delivery device comprises an elongate paper substrate disposed around a guidewire prior to insertion into the body chamber, and wherein the method comprises:
inserting the elongate paper substrate and guidewire into the body chamber;
releasing the elongate paper substrate from the guidewire within the body chamber; and
removing the guidewire from the body chamber.

11. The method of claim 10, wherein the body chamber has a spiraled shape, and wherein the method further comprises:
following insertion of the elongate paper substrate and guidewire into the body chamber, but prior to releasing the elongate paper substrate from the guidewire, allowing the guidewire and elongate paper substrate to accept a curved arrangement matching at least a portion of the spiral shape of the body chamber.

12. The method of claim 10, wherein releasing the elongate paper substrate from the guidewire within the body chamber comprises:
manipulating the guidewire to place at least a portion of the elongate paper substrate in contact with the surface of the body chamber.

13. (canceled)

14. The method of claim 1, wherein the therapeutic substance delivery device comprises a delivery substrate defining at least one cannula and one or more outlets fluidically coupling the at least one cannula to an exterior surface of the delivery substrate, and wherein delivering the one or more therapeutic substances to the body chamber comprises:
delivering the one or more therapeutic substances from a source external to the body chamber via the at least one cannula and the one or more outlets.

15. The method of claim 1, wherein the body chamber is located behind a tissue barrier in the recipient, and wherein the method further comprises:
forming an opening in the tissue barrier of the recipient;
inserting the therapeutic substance delivery device into the body chamber behind the tissue barrier via the opening; and
sealing the opening in the tissue barrier with the therapeutic substance delivery device within the body chamber.

16. An apparatus, comprising:
a fully bioresorbable substrate configured to be inserted into a body chamber of a recipient to assume a position in close proximity to a first side of the body chamber without affecting structures of the body chamber on a second side of the body chamber; and
one or more therapeutic substances disposed in or on the substrate.

17. The apparatus of claim 16, wherein the substrate is formed from an adhesive material configured to adhere to a wall at the first side of the body chamber.

18. The apparatus of claim 17, wherein the adhesive material comprises an adhesive gel.

19. The apparatus of claim 17, wherein the adhesive material is configured to adhere to a wet surface at the first side of the body chamber.

20. The apparatus of claim 16, wherein the substrate comprises a first surface configured to be positioned adjacent a wall at the first side of the body chamber, and wherein the apparatus comprises:
an adhesive disposed on the first surface of the substrate.

21. (canceled)

22. (canceled)

23. (canceled)

24. (canceled)

25. (canceled)

26. The apparatus of claim 25, wherein the width of the substrate is at least twice the thickness of the substrate.

27. (canceled)

28. The apparatus of claim 16, wherein the substrate has a thickness that is less than approximately 200 microns.

29. (canceled)

30. The apparatus of claim 16, wherein the substrate has a pre-curved shape and is configured to be held straight prior to insertion into the body chamber, wherein during insertion into the body chamber the substrate is configured to return to the pre-curved shape.

31. (canceled)

32. (canceled)

33. The apparatus of claim 16, wherein the substrate has an elongate length with a variable aspect ratio along the elongate length thereof.

34. The apparatus of claim 16, wherein the body chamber is a scala tympani of a cochlea of the recipient, wherein the first side of the body chamber is the modiolar wall, and wherein the substrate is configured to, following insertion, have a minimum spacing from a lateral wall of the cochlea.

35-48. (canceled)
